(19) United States
(12) Patent Application Publication
(10) Pub. No.: US 2022/0207799 A1
(43) Pub. Date: Jun. 30, 2022

(54) PSYCHOLOGICAL STATE VISUALIZATION APPARATUS, METHOD AND PROGRAM FOR THE SAME

(71) Applicant: NIPPON TELEGRAPH AND TELEPHONE CORPORATION, Tokyo (JP)

(72) Inventors: Junji WATANABE, Tokyo (JP); Aiko MURATA, Tokyo (JP); Reiko ARUGA, Tokyo (JP)

(73) Assignee: NIPPON TELEGRAPH AND TELEPHONE CORPORATION, Tokyo (JP)

(21) Appl. No.: 17/605,675

(22) PCT Filed: Apr. 25, 2019
(86) PCT No.: PCT/JP2019/017593

§ 371 Date: Oct. 22, 2021

(51) Int. Cl. G06T 11/20; G06N 3/08

(52) U.S. Cl. CPC G06T 11/206; G06N 3/08

(57) ABSTRACT

A technology is provided in which a temporal change of a psychological state is visualized in an intuitively easy-to-understand manner without utilizing a biosensor. A psychological state visualization apparatus includes: an input unit configured to receive, as an input, a psychological state sensibility representation word representing a psychological state of a user at a time point and a psychological state index value corresponding to the psychological state sensibility representation word and indicating the psychological state of the user; and a presentation unit configured to visualize and present a character string of the psychological state sensibility representation word and the psychological state index value in a form in which a temporal variation is visible.

### TECHNICAL FIELD

The present invention relates to a psychological state visualization apparatus, a method, and a program thereof that visualize a psychological state of a user.

### BACKGROUND ART

Patent Literature 1 is known as a related-art technology for an apparatus that visualizes a psychological state of a user. In Patent Literature 1, information acquired by a biosensor attached to a user is analyzed for each predetermined time interval to estimate and display an emotion in the time interval of the user.

### CITATION LIST

### Patent Literature

Patent Literature 1: JP-2016-106689 A

### SUMMARY OF THE INVENTION

### Technical Problem

In Patent Literature 1, a device for acquiring a biosensor needs to be attached to a body. In addition, there is a problem in that an emotion estimated based on data of the biosensor is sometimes inconsistent with the emotion of which the user himself/herself is aware and it is intuitively difficult to understand.

In view of these problems, an object of the present invention is to provide a technology for visualizing a temporal change in psychological state in an intuitively easy-to-understand manner without utilizing a biosensor.

### Means for Solving the Problem

To solve the above problems, according to an aspect of the present invention, a psychological state visualization apparatus includes: an input unit configured to receive, as an input, a psychological state sensibility representation word representing a psychological state of a user at a time point and a psychological state index value corresponding to the psychological state sensibility representation word and indicating the psychological state of the user; and a presentation unit configured to visualize and present a character string of the psychological state sensibility representation word and the psychological state index value in a form in which a temporal variation is visible.

To solve the above problems, according to another aspect of the present invention, a psychological state visualization apparatus includes: an input unit configured to receive, as an input, a psychological state sensibility representation word representing a psychological state of a user at a time point; a psychological state estimation unit configured to use a psychological state estimation model to estimate a psychological state index value corresponding to the psychological state sensibility representation word input in the input unit, the psychological state estimation model being configured to receive the psychological state sensibility representation word as an input and convert the psychological state sensibility representation word into the psychological state index value corresponding to the psychological state sensibility representation word; and a presentation unit configured to visualize and present a character string of the psychological state sensibility representation word and the psychological state index value in a form in which a temporal variation is visible.

### Effects of the Invention

According to the present invention, an effect is exhibited that a temporal change in psychological state can be visualized in an intuitively easy-to-understand manner without utilizing a biosensor.

### BRIEF DESCRIPTION OF DRAWINGS

FIG. 1 is a functional block diagram of a psychological state visualization apparatus according to a first embodiment.

FIG. 2 is a flowchart of an example of processing of the psychological state visualization apparatus according to the first embodiment.

FIG. 3 is a diagram illustrating an example of visualization.

FIG. 4 is a diagram illustrating an example of visualization.

FIG. 5 is a diagram illustrating an example of visualization.

FIG. 6 is a diagram illustrating an example of visualization.

FIG. 7 is a functional block diagram of a psychological state visualization apparatus according to a second embodiment.

FIG. 8 is a flowchart of an example of processing of the psychological state visualization apparatus according to the second embodiment.

FIG. 9 is a diagram illustrating an example of a table including training data.

### DESCRIPTION OF EMBODIMENTS

Hereinafter, embodiments of the present invention will be described. In the drawings used in the following description, the same reference signs are given to components having the same function or the steps of performing the same processing, and duplicate description is omitted.

### First Embodiment

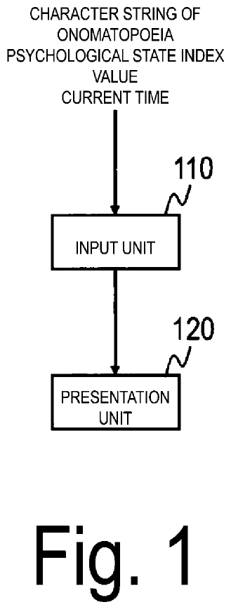

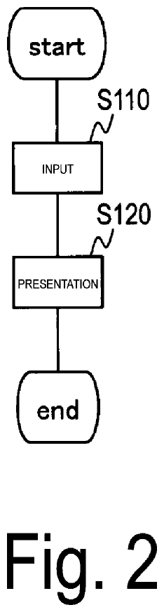

FIG. 1 is a functional block diagram of a psychological state visualization apparatus according to a first embodiment and FIG. 2 is a flowchart of processing thereof

The psychological state visualization apparatus includes an input unit 110 and a presentation unit 120.

The psychological state visualization apparatus receives, as an input, a character string of a psychological state sensibility representation word, a psychological state index value, and a corresponding time to visualize and present a time series of a psychological state. The psychological state sensibility representation word represents a psychological state of a user at a time point, and is a generic term of a word categorized, for example, in at least any of an onomatopoeia and an exclamation. The onomatopoeia is also a generic term of a word categorized, for example, in at least any of an onomatopoeic word, a mimetic word, and a psychomime. Here, an onomatopoeic word represents an actual sound using a speech sound, a mimetic word represents a non-sound sensation using a speech sound, and a psychomime represents a psychological state using a speech sound. Note that the exclamation is sometimes referred to as an interjection. Meanwhile, for example, the psychological state index value indicates a psychological state of a user and represents a degree of the psychological state at a time point. Hereinafter, a case in which the psychological state sensibility representation word is an onomatopoeia will be described, but the same processing can be performed in the case of the exclamation.

For example, the psychological state visualization apparatus is a special apparatus configured by reading a special program into a well-known or dedicated computer including a central processing unit (CPU), a main storage device (random access memory (RAM)), and the like. For example, the psychological state visualization apparatus executes each processing under control of the central processing unit. For example, data input to the psychological state visualization apparatus or data obtained in each processing is stored in the main storage device, and the data stored in the main storage device is read into the central processing unit and used in another processing as necessary. At least a part of each processing unit of the psychological state visualization apparatus may be configured with hardware such as an integrated circuit. Each storage unit included in the psychological state visualization apparatus can be configured with a main storage device such as a random access memory (RAM) or middleware such as a relational database or a key-value store. However, each storage unit does not need to be included inside the psychological state visualization apparatus and may be configured with an auxiliary storage device configured with a hard disk, an optical disk, or a semiconductor memory element such as a flash memory and may be included outside the psychological state visualization apparatus.

For example, the psychological state visualization apparatus may be implemented on a mobile terminal, a tablet terminal, or the like.

Each unit will be described below.

Input Unit 110

The input unit 110 receives, from a user, input of a character string of an onomatopoeia representing a current state of himself/herself, a psychological state index value at that time, and a corresponding time (S110), and outputs the input to the presentation unit 120.

Examples of the psychological state index value include:
(1) a value representing a positive degree of emotion in 9 levels, with a state of comfort as 4 and a state of discomfort as −4;
(2) a value representing a degree of joy in 5 levels, with a joyful state as 4 and a joyless state as 0; and
(3) a value representing a degree of motivation in 5 levels, with a motivated state as 4 and an out-of-motivation state as 0, and
   the psychological state index value is what represents a preset yardstick in a plurality of levels (9 or 5 levels in the examples described above).

One psychological state index value may be used (for example, any one of (1) to (3) described above), or a plurality of them (for example, (1) and (3)) may be used.

For example, an entry field of a character string of an onomatopoeia and an entry field of a psychological state index value are displayed on a display of a mobile terminal, a tablet terminal, or the like, and a user inputs a character string of an onomatopoeia and a psychological state index value via an input unit such as a touch panel.

Note that the entry field may have a configuration in which character strings of predetermined kinds of onomatopoeias and psychological state index values represented in a plurality of preset levels are displayed and selected, or may have a configuration in which a user freely inputs a character string or a psychological state index value.

The corresponding time may be a time at which a user inputs a character string of an onomatopoeia and a psychological state index value via an input unit such as a touch panel (input time), or a time at which the input unit 110 receives a character string of an onomatopoeia and a psychological state index value (receipt time). In the case of the input time, an input unit such as a touch panel may be configured to acquire a time from a built-in clock, an NTP server, or the like and output the time to the input unit 110, and in the case of the receipt time, the input unit 110 may be configured to acquire a receipt time from, for example, a built-in clock, an NTP server, or the like.

Presentation Unit 120

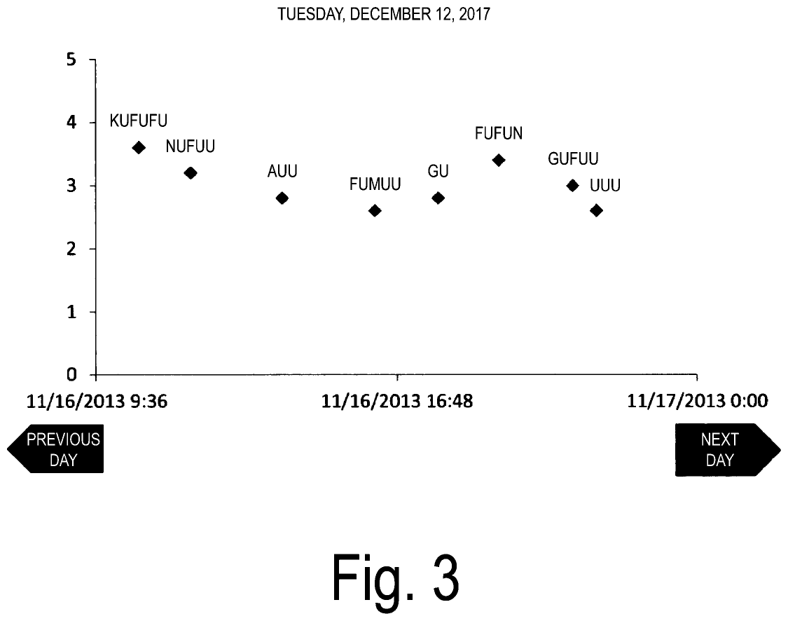

The presentation unit 120 receives, as an input, a character string of an onomatopoeia, a psychological state index value corresponding to the character string of the onomatopoeia, and a corresponding time, and visualizes and presents the character string of the onomatopoeia and the psychological state index value corresponding to the character string of onomatopoeia in a form in which a temporal variation is visible, on the basis of these pieces of information (S120). In the present embodiment, the presentation unit 120 displays, near a marker representing a psychological state index value (hereinafter, also referred to as a psychological state level) corresponding to a time, information on a character string of an onomatopoeia corresponding to the same time (see FIGS. 3 to 6).

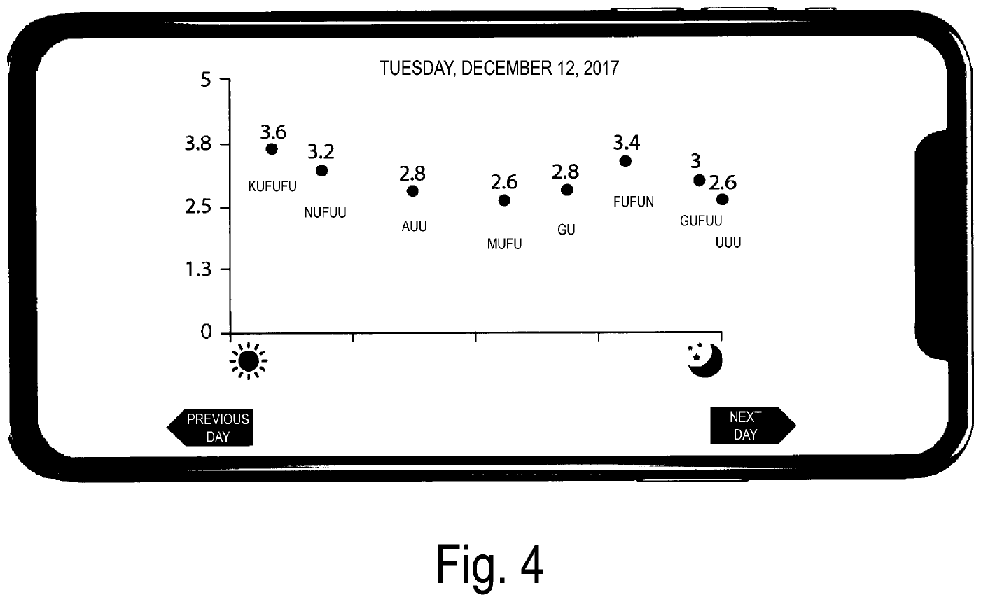

In a case where there is one kind of psychological state level to be presented (e.g., the degree of joy described above), the level is visualized in a two-dimensional graph with time as a horizontal axis and a psychological state level as a vertical axis, for example, as illustrated in FIG. 3 and FIG. 4. In this example, near a marker representing a psychological state level, a character string of an onomatopoeia corresponding to the same time is displayed. The time axis (horizontal axis) may be any type of axis, such as a form in which time information is displayed as illustrated in FIG. 3, or a form in which an icon corresponding to a time is displayed as illustrated in FIG. 4, as long as passage of time (time series) can be grasped. In other words, any type of axis may be used as long as a temporal variation of a character string of an onomatopoeia and a psychological state level is visible. In the example of FIG. 4, psychological state level values are also displayed near markers that visualize the temporal variation of the psychological state level.

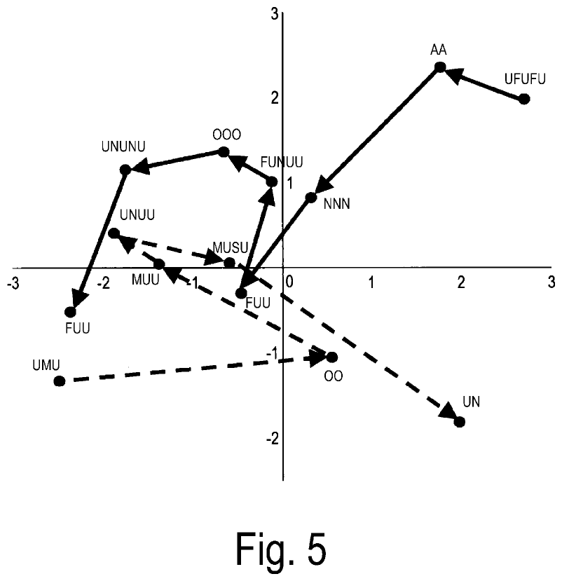

In a case where there are two kinds of psychological state levels to be presented, for example, as in FIG. 5, a vertical axis and a horizontal axis indicate different psychological state levels, a set of psychological state levels for each time is represented by a marker, and a marker at a certain time is used as a starting point and connected to a marker at a next time as an endpoint by an arrow to visualize a temporal change in the two kinds of psychological state levels over time. In other words, presentation is performed so that a temporal order of a character string of an onomatopoeia and two kinds of psychological state levels can be seen. Here, a solid arrow and a dashed arrow in FIG. 5 represent changes in psychological states of an identical person on different days.

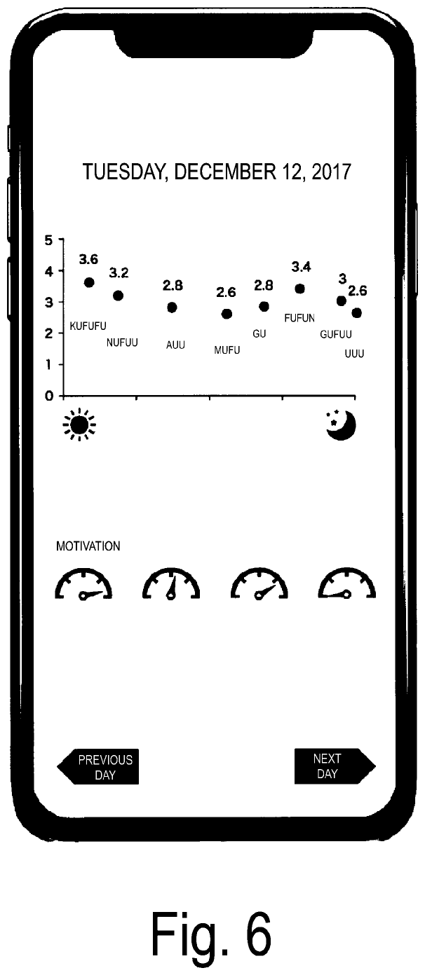

However, when a plurality of kinds of psychological state levels are associated, it is not necessary to represent all the levels in axes of a graph as in FIG. 5. For example, as illustrated in FIG. 6, a part of the psychological state levels (one or two kinds) may be used to perform a graph display as in FIGS. 3 to 5 (FIG. 4 in this example), and the remaining psychological state levels may be visualized by icons, graphs, or the like, respectively. In FIG. 6, “motivation” of the psychological state levels is visualized as an icon of an indicator in a manner different from the graph. In this example, one day is divided into four time zones, and a representative value of a motivation level corresponding to an onomatopoeia acquired in each time zone is represented by a scale mark of the indicator to visualize and present the temporal variation of the motivation in a visually graspable manner.

As described above, in the presentation unit, a temporal variation of at least one or more kinds of psychological state levels is visualized by a multi-dimensional graphic (graph), and a character string of an onomatopoeia only needs to be displayed near a position corresponding to each time in the graphic. Alternatively, sizes of at least one or more kinds of psychological state levels may be visualized and presented by being associated with a scale mark, length, or size.

Advantages

With such a configuration, a temporal change in a psychological state can be visualized in an intuitively easy-to-understand manner without utilizing a biosensor.

Modifications

In the present embodiment, although a case has been described in which the psychological state visualization apparatus is implemented on a mobile terminal, a tablet terminal, or the like, for example, the psychological state visualization apparatus may be implemented on a server having a mobile terminal, a tablet terminal, or the like as a client. In this case, the presentation unit 120 outputs information regarding visualization information to be presented, and presents the information on a display of a mobile terminal, a tablet terminal, or the like. The information regarding visualization information to be presented may be, for example, an image to be presented, or a parameter used in generating an image to be presented on a mobile terminal, a tablet terminal, or the like.

### Second Embodiment

Parts different from the first embodiment will be mainly described.

In the first embodiment, a user also inputs a psychological state index value with a character string of an onomatopoeia hourly in the input unit 110, while in a second embodiment, a user inputs only a character string of an onomatopoeia. The second embodiment is different from the first embodiment in that a component to estimate a psychological state index value corresponding to a character string of an onomatopoeia input by a user is added.

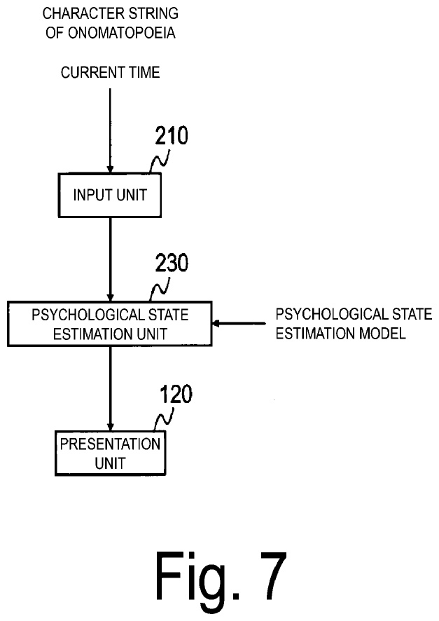

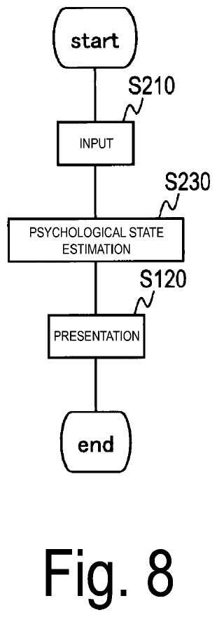

FIG. 7 is a functional block diagram of the psychological state visualization apparatus according to the first embodiment, and FIG. 8 is a flowchart of processing thereof

The psychological state visualization apparatus includes an input unit 210, a psychological state estimation unit 230, and a presentation unit 120.

Input Unit 210

The input unit 210 receives, from a user, input of a character string of an onomatopoeia representing a current state of himself/herself and a corresponding time (S210), outputs the character string of the onomatopoeia to the presentation unit 120 and the psychological state estimation unit 230, and outputs the corresponding time to the presentation unit 120. A receipt method is the same as that in the first embodiment except that there is no psychological state index value.

Psychological State Estimation Unit 230

The psychological state estimation unit 230 receives a character string of an onomatopoeia as an input, uses a psychological state estimation model to estimate a psychological state index value corresponding to the onomatopoeia (S230), and outputs the estimated value to the presentation unit 120.

The psychological state estimation model is a model that converts the input character string of the onomatopoeia into a psychological state index value corresponding to the onomatopoeia, and is prepared in advance using data (training data) in which character strings of onomatopoeias acquired from a plurality of persons are associated with psychological state index values.

As the training data, data such as the data input to the input unit 110 in the first embodiment may be gathered from a plurality of persons to be used. At this time, the corresponding time may be removed. That is, a large amount of combinations of character strings of onomatopoeias representing psychological states of a user and psychological state index values at that time may be prepared and used as the training data.

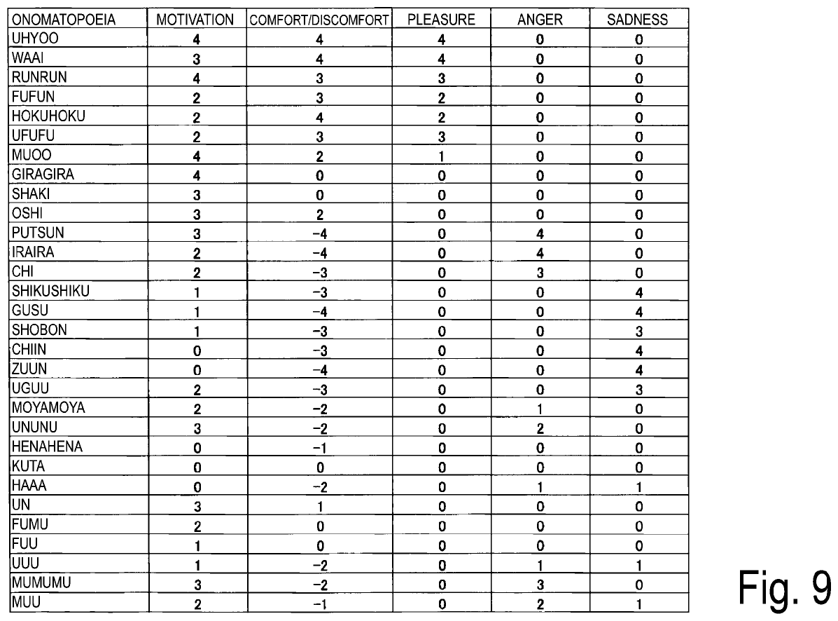

FIG. 9 illustrates an example of a table including the training data. In this example, motivation, pleasure, anger, and sadness are represented as a numerical value in five levels of 0 to 4, and the higher the respective degrees, the greater the numerical values. The comfort/discomfort is represented as a numerical value in 9 levels of −4 to 4, and the higher the degree of “comfort”, the greater the positive value, the higher the degree of discomfort, the greater the negative value.

### EXAMPLE 1 OF PSYCHOLOGICAL STATE ESTIMATION MODEL

Association of onomatopoeias (character strings) and psychological state index values corresponding thereto (e.g., a table and a list) is used as the psychological state estimation model. As each of the psychological state index values in a table or a list, for example, a representative value (an average value, a median value, or the like) of the psychological state index values assigned to a certain onomatopoeia in the training data by respective persons is used.

### EXAMPLE 2 OF PSYCHOLOGICAL STATE ESTIMATION MODEL

In this example, the psychological state estimation model is a model trained by machine learning of a neural network or the like, based on onomatopoeias for training and the corresponding psychological state index values for training. For example, a neural network that receives an onomatopoeia (a character string) as an input and outputs a psychological state index value corresponding to the onomatopoeia is used as the psychological state estimation model. In this case, a parameter of the neural network is repeatedly updated so that an estimation result of a psychological state index value obtained by inputting an onomatopoeia (a character string) in the training data to the neural network in which a proper initial value has been set in advance approaches the psychological state index value associated with the onomatopoeia in the training data, thereby training the psychological state estimation model. Note that in a case where training data in which a plurality of psychological state index values are input for one onomatopoeia is used, the psychological state estimation model may be trained so that the output of the psychological state estimation model is also a list (set) of a plurality of psychological state index values.

Advantages

With the above configuration, the same advantageous effects as those of the first embodiment can be achieved.

Further, it is possible to spare a user the trouble of inputting a psychological state index value.

Modifications

The description has been made that a user inputs a character string of an onomatopoeia in the input unit of the first embodiment or the second embodiment, but the present invention is not limited to the input of a character string itself.

For example, an illustration, an image, or the like associated with an onomatopoeia in a one-to-one basis may be input. In this case, a database in which onomatopoeias are associated with illustrations, images, or the like may be provided, the input unit may receive an illustration, an image, or the like as an input, and a character string of the corresponding onomatopoeia may be retrieved from the database and output to the presentation unit 120.

Alternatively, for example, a character string of an onomatopoeia included in a speech recognition result of an utterance of a user may be automatically extracted to receive the input of the character string of the onomatopoeia. For example, the input unit may receive a speech signal in place of a character string of an onomatopoeia as an input, perform speech recognition processing with a speech recognition unit (not illustrated), obtain a speech recognition result, extract a character string of an onomatopoeia from the obtained result, and output the extracted character string to the presentation unit 120. For example, a database in which character strings of onomatopoeias of interest are stored is provided, and a character string of an onomatopoeia is extracted from a speech recognition result by referring to the database.

Furthermore, in the second embodiment, it is not necessary to receive a psychological state index value as an input, and thus, for example, a character string of an onomatopoeia automatically extracted from character strings in a text input when a user composes a mail or creates a comment for posting to the Web may be used as an input, or a character string of an onomatopoeia automatically extracted from a speech recognition result of voice of a user when the user is talking on a mobile phone or the like may be used as an input.

Other Modifications

The present invention is not limited to the above embodiments and modifications. For example, the various processes described above may be executed not only in chronological order as described but also in parallel or on an individual basis as necessary or depending on the processing capabilities of the apparatuses that execute the processing. In addition, appropriate changes can be made without departing from the spirit of the present invention.

Program and Recording Medium

The various processing functions of each device (or apparatus) described in the above embodiments and modifications may be implemented by a computer. In this case, the processing details of the functions that each device may have are described in a program. When the program is executed by a computer, the various processing functions of the device are implemented on the computer.

The program in which the processing details are described can be recorded on a computer-readable recording medium. The computer-readable recording medium can be any type of medium such as a magnetic recording device, an optical disc, a magneto-optical recording medium, or a semiconductor memory.

The program is distributed, for example, by selling, giving, or lending a portable recording medium such as a DVD or a CD-ROM with the program recorded on it. The program may also be distributed by storing the program in a storage device of a server computer and transmitting the program from the server computer to another computer through a network.

For example, a computer configured to execute such a program first stores, in its storage unit, the program recorded on the portable recording medium or the program transmitted from the server computer. Then, the computer reads the program stored in its storage unit and executes processing in accordance with the read program. In a different embodiment of the program, the computer may read the program directly from the portable recording medium and execute processing in accordance with the read program. The computer may also sequentially execute processing in accordance with the program transmitted from the server computer each time the program is received from the server computer. In another configuration, the processing may be executed through a so-called application service provider (ASP) service in which functions of the processing are implemented just by issuing an instruction to execute the program and obtaining results without transmission of the program from the server computer to the computer. The program includes information that is provided for use in processing by a computer and is equivalent to the program (such as data having properties defining the processing executed by the computer rather than direct commands to the computer).

In this mode, the device is described as being configured by executing the predetermined program on the computer, but at least a part of the processing may be implemented in hardware.

1. A psychological state visualization apparatus, comprising:
an input receiver configured to receive, as an input, a psychological state sensibility representation word representing a psychological state of a user at a time point and a psychological state index value corresponding to the psychological state sensibility representation word and indicating the psychological state of the user; and
a presenter configured to visually present a character string of the psychological state sensibility representation word and the psychological state index value in a form in which a temporal variation is visible.

2. A psychological state visualization apparatus, comprising:
an input receiver configured to receive a psychological state sensibility representation word representing a psychological state of a user at a time point;
a psychological state determine configured to, using a psychological state estimation model, determine a psychological state index value corresponding to the psychological state sensibility representation word, the psychological state estimation model being configured to receive the psychological state sensibility representation word as an input and convert the psychological state sensibility representation word into the psychological state index value corresponding to the psychological state sensibility representation word; and
a presenter configured to visually present a character string of the psychological state sensibility representation word and the psychological state index value in a form in which a temporal variation is visible.

3. The psychological state visualization apparatus according to claim 2, wherein the psychological state estimation model is a model in which a psychological state sensibility representation word is associated with a psychological state index value corresponding to the psychological state sensibility representation word.

4. The psychological state visualization apparatus according to claim 2, wherein the psychological state estimation model is a model trained by machine learning based on a psychological state sensibility representation word for training and a psychological state index value for training corresponding to the psychological state sensibility representation word.

5. (canceled)

6. A psychological state visualization method, comprising:
receiving, by a psychological state determiner, as an input, a psychological state sensibility representation word representing a psychological state of a user at a time point;
determining, by a psychological state determiner using, a psychological state estimation model, a psychological state index value corresponding to the psychological state sensibility representation word input, the psychological state estimation model being configured to receive the psychological state sensibility representation word and convert the psychological state sensibility representation word into the psychological state index value corresponding to the psychological state sensibility representation word; and
visually presenting, by a presenter, a character string of the psychological state sensibility representation word and the psychological state index value in a form in which a temporal variation is visible.

7. (canceled)

8. The psychological state visualization apparatus according to claim 2, wherein the psychological state estimation model includes a neural network receiving a character string among input and providing a psychological state index value as output.

9. The psychological state visualization method according to claim 6, wherein the psychological state estimation model includes a neural network receiving a character string among input and providing a psychological state index value as output.

10. The psychological state visualization method according to claim 6, wherein the psychological state estimation model is a model in which a psychological state sensibility representation word is associated with a psychological state index value corresponding to the psychological state sensibility representation word.

11. The psychological state visualization method according to claim 6, wherein the psychological state estimation model is a model trained by machine learning based on a psychological state sensibility representation word for training and a psychological state index value for training corresponding to the psychological state sensibility representation word.
